(19) United States
(12) Patent Application Publication
(10) Pub. No.: US 2006/0034865 A1
(43) Pub. Date: Feb. 16, 2006

(54) Soluble MHC artificial antigen presenting cells

(76) Inventors: William H. Hildebrand, Edmond, OK (US); Heather D. Hickman, Oklahoma City, OK (US)

Correspondence Address: DUNLAP, CODDING & ROGERS P.C., PO BOX 16370, OKLAHOMA CITY, OK, 73113, US

(21) Appl. No.: 11/253,009

(22) Filed: Oct. 18, 2005

Related U.S. Application Data
(63) Continuation of application No. 10/050,231, filed on Jan. 16, 2002, now abandoned.
(63) Continuation-in-part of application No. 10/022,066, filed on Dec. 18, 2001, now abandoned.
(60) Provisional application No. 60/261,978, filed on Jan. 16, 2001.

(51) Int. Cl. A61K 39/00; A61K 9/127

(52) U.S. Cl. 424/192.1; 424/450

(57) ABSTRACT

An artificial antigen presenting cell includes a liposome having at least one recombinant soluble MHC-peptide complex incorporated therein. The artificial antigen presenting cell may also include at least one additional signal molecule incorporated therein for manipulating the intensity and quality of the immune response. The recombinant soluble MHC molecule is obtained by a method utilizing PCR amplification of gDNA or cDNA, and a tag is attached thereto for anchoring the recombinant soluble MHC molecule to the liposome.

### CROSS REFERENCE TO RELATED APPLICATIONS

This application is a continuation of U.S. Ser. No. 10/050,231, filed Jan. 16, 2002, entitled “SOLUBLE MHC ARTIFICIAL ANTIGEN PRESENTING CELLS”, which claims priority under 35 U.S.C. § 119(e) of provisional U.S. Ser. No. 60/261,978, filed Jan. 16, 2001, entitled “SOLUBLE HLA ARTIFICIAL ANTIGEN PRESENTING CELLS”, the contents of which are hereby expressly incorporated in their entirety by reference. This application is also a continuation-in-part of U.S. Ser. No. 10/022,066, filed Dec. 18, 2001, entitled “METHOD AND APPARATUS FOR THE PRODUCTION OF SOLUBLE MHC ANTIGENS AND USES THEREOF”, the contents of which are hereby expressly incorporated in their entirety by reference.

### STATEMENT REGARDING FEDERALLY FUNDED RESEARCH

Not Applicable.

### BACKGROUND OF THE INVENTION

1. Field of the Invention

The field of the invention relates in general to carrier molecules that display MHC-peptide complexes for T cell binding and activation and more particularly, but not by way of limitation, to artificial antigen presenting cells that have individual MHC-peptide complexes incorporated therein.

2. Brief Description of the Background Art

Class I major histocompatibility complex (MHC) molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as products of bacteria that multiply outside of cells, wherein such products include protein toxins secreted by the bacteria that often times have deleterious and even lethal effects on the host (e.g. human). In this manner, class II molecules convey information regarding the fitness of the extracellular space in the vicinity of the cell displaying the class II molecule to immune effector cells, including but not limited to, CD4⁺ helper T cells, thereby helping to eliminate such pathogens the examination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events; as such, hundreds of different HLA types exist throughout the world's population, resulting in a large immunological diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, large quantities of pure isolated HLA proteins are required in order to effectively study transplantation, autoimmunity disorders, and for vaccine development.

Since every individual has differing MHC molecules, the testing of numerous individual MHC molecules is a prerequisite for understanding the differences in disease susceptibility between individuals. Therefore, purified MHC molecules representative of the hundreds of different HLA types existing throughout the world's population are highly desirable for unraveling disease susceptibilities and resistances, as well as for designing therapeutics such as vaccines.

Class I HLA molecules alert the immune response to disorders within host cells. Peptides, which are derived from viral- and tumor-specific proteins within the cell, are loaded into the class I molecule's antigen binding groove in the endoplasmic reticulum of the cell and subsequently carried to the cell surface. Once the class I HLA molecule and its loaded peptide ligand are on the cell surface, the class I molecule and its peptide ligand are accessible to cytotoxic T lymphocytes (CTL). CTL survey the peptides presented by the class I molecule and destroy those cells harboring ligands derived from infectious or neoplastic agents within that cell.

While specific CTL targets have been identified, little is known about the breadth and nature of ligands presented on the surface of a diseased cell. From a basic science perspective, many outstanding questions have permeated through the art regarding peptide exhibition. For instance, it has been demonstrated that a virus can preferentially block expression of HLA class I molecules from a given locus while leaving expression at other loci intact. Similarly, there are numerous reports of cancerous cells that fail to express class I HLA at particular loci. However, there is an absence in the art as it presently stands of data describing how (or if) the three classical HLA class I loci differ in the immunoregulatory ligands they bind. It is therefore unclear in the art as it presently stands as to how class I molecules from the different loci vary in their interaction with viral- and tumor-derived ligands and the number of peptides each will present.

Discerning virus and tumor specific ligands for CTL recognition is an important component of vaccine design. Ligands unique to tumorigenic or infected cells can be tested and incorporated into vaccines designed to evoke a protective CTL response. Several methodologies are currently employed to identify potentially protective peptide ligands. One approach uses T cell lines or clones to screen for biologically active ligands among chromatographic fractions of eluted peptides. (Cox et al., Science, vol 264, 1994, pages 716-719, which is expressly incorporated herein by reference in its entirety) This approach has been employed to identify peptides ligands specific to cancerous cells. A second technique utilizes predictive algorithms to identify peptides capable of binding to a particular class I molecule based upon previously determined motif and/or individual ligand sequences. (De Groot et al., Emerging Infectious Diseases, (7) 4, 2001, which is expressly incorporated herein by reference in its entirety) Peptides having high predicted probability of binding from a pathogen of interest can then be synthesized and tested for T cell reactivity in precursor, tetramer or ELISpot assays.

However, there has been no readily available source of individual HLA molecules. The quantities of HLA protein available to those of ordinary skill in the art have been small and typically consisted of a mixture of different HLA molecules. Production of HLA molecules traditionally involves the growth and lysis of cells expressing multiple HLA molecules. Ninety percent of the population is heterozygous at each of the HLA loci; codominant expression results in multiple HLA proteins expressed at each HLA locus. To purify native class I or class II molecules from mammalian cells requires time-consuming and cumbersome purification methods, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of results cannot directly distinguish between the different HLA molecules, and one cannot be certain that any particular HLA molecule is responsible for a given result. Therefore, a need exists in the art for a method of producing substantial quantities of individual HLA class I or class II molecules so that they can be readily purified and isolated independent of other HLA class I or class II molecules. Such individual HLA molecules, when provided in sufficient quantity and purity, would provide a powerful tool for studying and measuring immune responses.

While tetramer technology provides an excellent method of identifying and assessing the immunogenicity of putative antigenic peptides in vitro, it is unable to produce an antigenic response in vivo and therefore is not useful in vaccine development or immunomodulation strategies. To achieve an immune response, not only is a stable interaction between antigen-presenting cells, that is, cells expressing MHC having the antigenic peptide bound therein, and T cells dependent on the absolute affinity between the T cell receptor and the MHC-antigenic peptide complex but also on the relative density of molecules available for contact at the interaction site. The proper density of MHC-antigenic peptide complexes is obtained by migration of such molecules toward the initial interaction site through a phenomenon known as “capping”, thereby forming what is known as the “immune synapse”, the machinery required for T-cell signaling.

The tetramer molecules, while expressing multiple copies of the MHC-antigenic peptide complexes, have a strained conformation that do not allow such complexes to move or migrate in such a fashion that can mimic the capping phenomenon, and therefore this technology is only useful in detection, rather than manipulation, of immune responses. However, Prakken et al (Nature Medicine (2000) 6:1406), the disclosure of which is expressly incorporated herein by reference, describes a system that mimics the physiological interactions between antigen presenting cells (cells expressing MHC) and T cells. Such system utilizes artificial antigen presenting cells (aAPC), which comprise a liposome having MHC molecules incorporated therein, and such aAPCs allow free movement of the MHC-peptide complexes in the artificial membrane. Such aAPCs are functional cell equivalents and allow molecules to move in the lipid bilayer, and do not possess the disadvantages and defects of mutated and altered cells which may contain other components which generate undesired responses when utilized for vaccine development or immunomodulation. However, Prakken et al only disclose two MHC molecules utilized in purified, native form from a B cell lymphoma which have been incorporated in the aAPC, and Prakken et al's method faces the same disadvantages and defects described above for the prior art, that is, the method would require isolating individual MHC molecules from hundreds of different, typed cell lines using time-consuming and cumbersome purification methods.

Therefore, there exists a need in the art for an improved system that more closely mimics the physiological interactions among T cells and antigen presenting cells. The present invention solves this need by coupling the production of individual soluble MHC molecules with an artificial antigen presenting cell methodology.

### BRIEF DESCRIPTION OF THE SEVERAL VIEWS OF THE DRAWINGS

FIG. 1 is a pictorial representation of an artificial antigen presenting cell constructed in accordance with the present invention.

FIG. 2 is a flow chart of the method of producing soluble MHC molecules in accordance with the present invention.

FIG. 3 is a flow chart of the epitope discovery of C-terminal-tagged soluble MHC molecules. Class I positive transfectants are infected with a pathogen of choice and soluble MHC preferentially purified utilizing the tag. Subtractive comparison of MS ion maps yields ions present only in infected cell, which are then MS/MS sequenced to derive class I epitopes.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. Also, it is to be understood that the phraseology and terminology employed herein is for the purpose of description and should not be regarded as limiting.

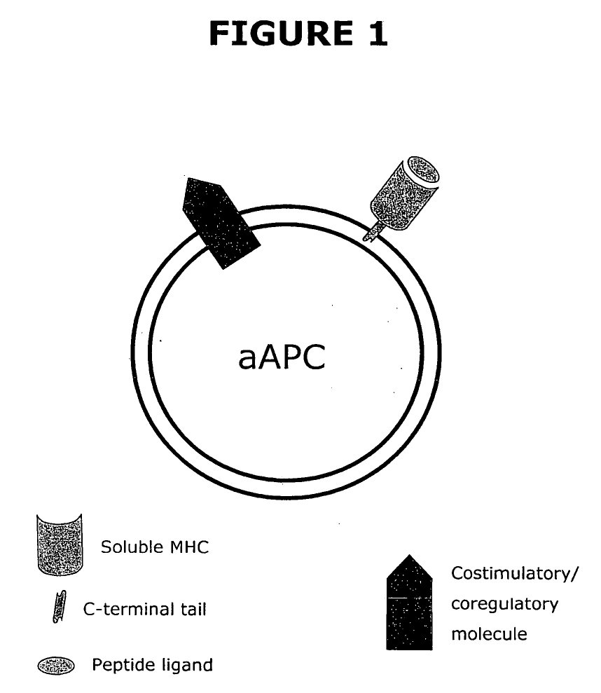

The present invention generally relates to a complex formed of a liposome having at least one recombinant soluble MHC-peptide complex incorporated therein such that the at least one recombinant soluble MHC-peptide complex is available to bind a T cell receptor on a T cell, thereby activating or suppressing such T cell. The unique liposome/recombinant soluble MHC-peptide complex of the present invention is referred to as an artificial antigen presenting cell (aAPC) and is graphically illustrated in FIG. 1. The recombinant soluble MHC-peptide complex of the aAPC includes a recombinant soluble MHC molecule (such as a Class I or II MHC molecule) containing a tag for anchoring the recombinant soluble MHC molecule to the liposome, and a peptide bound to an antigen binding groove of the recombinant soluble MHC molecule. The complex may further include at least one additional signal molecule incorporated in the liposome for manipulating the intensity and quality of the T cell response.

The purpose of the artificial antigen presenting cell (aAPC) is to specifically stimulate or mute a T cell driven immune response. T cells direct their immune response by targeting particular peptide ligands bound by particular MHC molecules. The aAPC of the present invention will have only the desired soluble MHC loaded with the desired peptide ligand(s). In this manner only T cells specific for the MHC-peptide complex on the aAPC will be stimulated/down-regulated. Placement of at least one additional costimulatory or coregulatory molecule on the aAPC will further manipulate the immune response; however, specificity of the aAPC is dictated by the soluble MHC-peptide complex.

The soluble MHC is anchored in the aAPC with a C-terminal tag specific for the aAPC surface. T cells can interact with the N-terminal portion of the soluble MHC and the peptide ligand bound thereto. Co receptor(s), if present, are similarly positioned in the aAPC.

The tag of the recombinant soluble MHC molecule may be a histidine tail or a biotinylation signal peptide, although other methods of tagging the soluble MHC molecules may be apparent to those of ordinary skill in the art and are, as such, within the scope of the present invention disclosed and claimed herein. When the tag is a histidine tail, nickel is disposed in the liposome so that the interaction between the nickel and the histidine tail maintains the recombinant soluble MHC molecule in an anchored position on the liposome. When the tag is a biotinylation signal peptide, the recombinant soluble MHC molecule containing the biotinylation signal peptide is biotinylated, and streptavidin is disposed in the liposome so that the interaction between biotin and the streptavidin maintains the recombinant soluble MHC molecule in an anchored position on the liposome. However, it is to be understood that the tag of the recombinant soluble MHC molecule is not limited to the embodiments described herein above, and one of ordinary skill in the art can envision other tags that may be utilized in accordance with the present invention.

In addition, the tag may also facilitate in purification of the soluble MHC molecules produced therefrom as well as anchoring the recombinant soluble MHC molecule to the liposome. For example, the use of either the histidine tail or the biotinylation signal peptide as the tag would allow purification of the soluble MHC molecules via a nickel or streptavidin column, respectively.

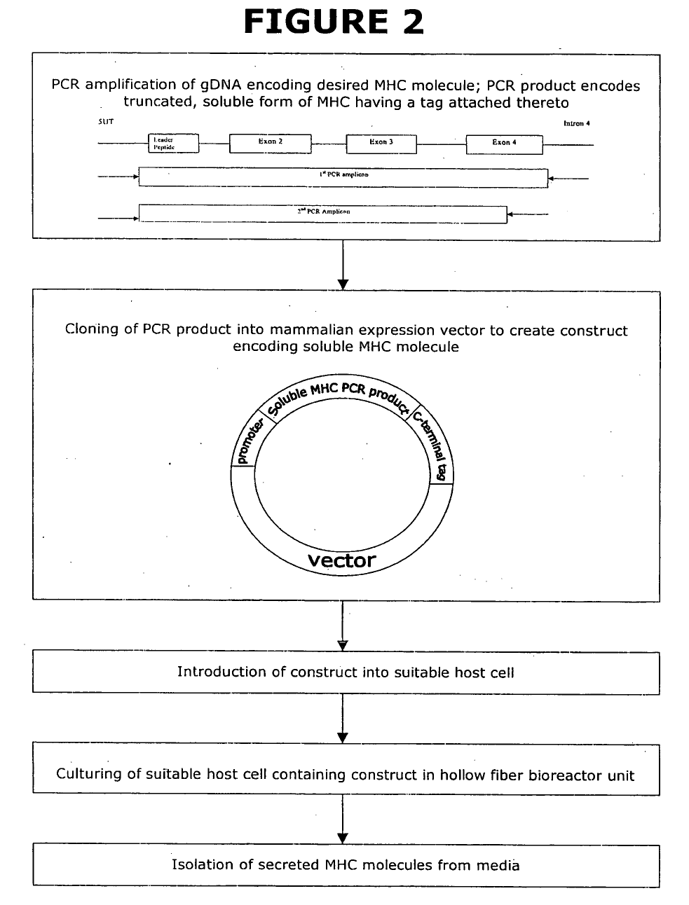

The present invention envisions a method of producing MHC molecules coupled with artificial antigen presenting cell technology to produce a complex comprising a liposome having at least one recombinant soluble MHC-peptide complex incorporated therein. The method of producing MHC molecules is described in detail in copending application U.S. Ser. No. 10/022,066, filed Dec. 18, 2001, entitled “METHOD AND APPARATUS FOR THE PRODUCTION OF SOLUBLE MHC ANTIGENS AND USES THEREOF”, the Specification of which is hereby specifically incorporated in its entirety by reference. Such method is summarized in FIG. 2.

In the method of producing soluble MHC molecules disclosed in U.S. Ser. No. 10/022,066, MHC molecules are secreted from mammalian cells in a bioreactor unit, and substantial quantities of individual MHC molecules are obtained by modifying class I or class II molecules so they are secreted. Secretion of soluble MHC molecules overcomes the disadvantages and defects of the prior art in relation to the quantity and purity of MHC molecules produced. Problems of quantity are overcome because the cells producing the MHC do not need to be detergent lysed or killed in order to obtain the MHC molecule. In this way the cells producing secreted MHC remain alive and therefore continue to produce MHC. Problems of purity are overcome because the only MHC molecule secreted from the cell is the one that has specifically been constructed to be secreted. Thus, transfection of vectors encoding such secreted MHC molecules into cells which may express endogenous, surface bound MHC provides a method of obtaining a highly concentrated form of the transfected MHC molecule as it is secreted from the cells. Greater purity can be assured by transfecting the secreted MHC molecule into MHC deficient cell lines.

Production of the MHC molecules in a hollow fiber bioreactor unit allows cells to be cultured at a density substantially greater than conventional liquid phase tissue culture permits. Dense culturing of cells secreting MHC molecules further amplifies the ability to continuously harvest the transfected MHC molecules. Dense bioreactor cultures of MHC secreting cell lines allow for high concentrations of individual MHC proteins to be obtained. Highly concentrated individual MHC proteins provide an advantage in that most downstream protein purification strategies perform better as the concentration of the protein to be purified increases. Thus, the culturing of MHC secreting cells in bioreactors allows for a continuous production of individual MHC proteins in a concentrated form.

The method of producing MHC molecules utilized in the present invention begins by obtaining genomic DNA (gDNA) or complementary DNA (cDNA) which encodes the desired MHC class I or class II molecule. Alleles at the locus which encode the desired MHC molecule are PCR amplified in a locus specific manner utilizing at least one locus-specific primer. These locus specific PCR products may include the entire coding region of the MHC molecule or a portion thereof. That is, the PCR reaction may be carried out in such a manner that the coding regions encoding the cytoplasmic and transmembrane domains of the MHC allele are not amplified, and therefore the PCR product produced therefrom encodes a truncated, soluble form of the MHC molecule that will be secreted rather than anchored to the cell surface. In one embodiment a nested or hemi-nested PCR is applied to produce a truncated form of the class I or class II gene. In another embodiment the PCR will directly truncate the MHC molecule, for example, by use of a locus-specific 3′ primer having a stop codon incorporated therein.

Locus specific PCR products are cloned into a mammalian expression vector and screened with a variety of methods to identify a clone encoding the desired MHC molecule. The cloned MHC molecules are DNA sequenced to ensure fidelity of the PCR. Faithful truncated clones of the desired MHC molecule are then introduced by transfection or electroporation into at least one suitable host cell, such as a mammalian cell line. The suitable host cell is then cultured under conditions that allow for expression of recombinant soluble MHC molecules from the construct. Such recombinant soluble MHC molecules produced in this manner are folded naturally and are trafficked through the cell in such a manner that they are identical in functional properties to a native MHC molecule expressed from the MHC allele and thereby bind peptide ligands in an identical manner as full-length, cell-surface-expressed MHC molecules. Such culture conditions also allow for endogenous loading of a peptide ligand into the antigen binding groove of each soluble MHC molecule prior to secretion of the soluble MHC molecule from the cell. Therefore, recombinant soluble MHC-peptide complexes can be isolated from the media.

The host cell containing the construct encoding the recombinant soluble class I MHC molecule may either lack endogenous class I MHC molecule expression or express endogenous class I MHC molecules. One of ordinary skill in the art would note the importance, given the present invention, that cells expressing endogenous class I MHC molecules may spontaneously release MHC into solution upon natural cell death. In cases where this small amount of spontaneously released MHC is a concern, the transfected class I MHC molecule can be “tagged” such that it can be specifically purified away from spontaneously released endogenous class I molecules in cells that express class I molecules. For example, a DNA fragment encoding a Histidine tail may be attached to the DNA encoding the protein by the PCR reaction or may be encoded by the vector into which the PCR fragment is cloned, and such Histidine tail, therefore, further aids in the purification of the class I MHC molecules away from endogenous class I molecules. Tags beside a histidine tail have also been demonstrated to work, and one of ordinary skill in the art of tagging proteins for downstream purification would appreciate and know how to tag a MHC molecule in such a manner so as to increase the ease by which the MHC molecule may be purified. In addition, such a tag may serve two purposes: besides allowing for purification of the recombinant MHC molecule, the tag may further be utilized in anchoring the recombinant soluble MHC molecule to a liposome, as will be discussed in greater detail herein below.

Cloned genomic DNA fragments contain both exons and introns as well as other non-translated regions at the 5′ and 3′ termini of the gene. Following transfection into a cell line which transcribes the genomic DNA (gDNA) into RNA, cloned genomic DNA results in a protein product thereby removing introns and splicing the RNA to form messenger RNA (mRNA), which is then translated into an MHC protein. Transfection of MHC molecules encoded by gDNA therefore facilitates reisolation of the gDNA, mRNA/cDNA, and protein. Production of MHC molecules in non-mammalian cell lines such as insect and bacterial cells requires cDNA clones, as these lower cell types do not have the ability to splice introns out of RNA transcribed from a gDNA clone. In these instances the mammalian gDNA transfectants of the present invention provide a valuable source of RNA which can be reverse transcribed to form MHC cDNA. The cDNA can then be cloned, transferred into cells, and then translated into protein. In addition to producing secreted MHC, such gDNA transfectants therefore provide a ready source of mRNA, and therefore cDNA clones, which can then be transfected into non-mammalian cells for production of MHC. Thus, the present invention which starts with MHC genomic DNA clones allows for the production of MHC in cells from various species.

A key advantage of starting from gDNA is that viable cells containing the MHC molecule of interest are not needed. Since all individuals in the population have a different MHC repertoire, one would need to search more than 500,000 individuals to find someone with the same MHC complement as a desired individual—such a practical example of this principle is observed when trying to find a donor to match a recipient for bone marrow transplantation. Thus, if it is desired to produce a particular MHC molecule for use in an experiment or diagnostic, a person or cell expressing the MHC allele of interest would first need to be identified. Alternatively, in the method of the present invention, only a saliva sample, a hair root, an old freezer sample, or less than a milliliter (0.2 ml) of blood would be required to isolate the gDNA. Then, starting from gDNA, the MHC molecule of interest could be obtained via a gDNA clone as described herein, and following transfection of such clone into mammalian cells, the desired protein could be produced directly in mammalian cells or from cDNA in several species of cells using the methods of the present invention described herein.

Current experiments to obtain an MHC allele for protein expression typically start from mRNA, which requires a fresh sample of mammalian cells that express the MHC molecule of interest. Working from gDNA does not require gene expression or a fresh biological sample. It is also important to note that RNA is inherently unstable and is not as easily obtained as is gDNA. Therefore, if production of a particular MHC molecule starting from a cDNA clone is desired, a person or cell line that is expressing the allele of interest must traditionally first be identified in order to obtain RNA. Then a fresh sample of blood or cells must be obtained; experiments using the methodology of the present invention show that ≧5 milliliters of blood that is less than 3 days old is required to obtain sufficient RNA for MHC cDNA synthesis. Thus, by starting with gDNA obtained from a sample such as blood, saliva, hair, semen, or sweat, the breadth of MHC molecules that can be readily produced is expanded. This is a key factor in a system as polymorphic as the MHC system; hundreds of MHC molecules exist, and not all MHC molecules are readily available. This is especially true of MHC molecules unique to isolated populations or of MHC molecules unique to ethnic minorities. Starting class I or class II MHC molecule expression from the point of genomic DNA simplifies the isolation of the gene of interest and insures a more equitable means of producing MHC molecules for study; otherwise, one would be left to determine whose MHC molecules are chosen and not chosen for study, as well as to determine which ethnic population from which fresh samples cannot be obtained and therefore should not have their MHC molecules included in a diagnostic assay.

While cDNA may be substituted for genomic DNA as the starting material, production of cDNA for each of the desired MHC class I types will require hundreds of different, MHC typed, viable cell lines, each expressing a different MHC class I type. Alternatively, fresh samples are required from individuals with the various desired MHC types. The use of genomic DNA as the starting material allows for the production of clones for many MHC molecules from a single genomic DNA sequence, as the amplification process can be manipulated to mimic recombinatorial and gene conversion events. Several mutagenesis strategies exist whereby a given class I gDNA clone could be modified at either the level of gDNA or at the cDNA resulting from this gDNA clone. The process of producing MHC molecules utilized in the present invention does not require viable cells, and therefore the degradation which plagues RNA is not a problem.

The soluble class I MHC proteins produced by the method described herein is utilized in production of artificial antigen presenting cells (aAPCs). The artificial antigen presenting cells of the present invention are complexes comprising at least one recombinant, soluble MHC-peptide complex isolated by the above described method and incorporated into a liposome. Liposomes are microscopic synthetic spheres of defined size and composition that are comprised of a membrane of lipid molecules (bilayer) surrounding an aqueous core. Other types of artificial antigen presenting cells have been developed, such as those based on mammalian cells (such as the human lymphoid hybrid T2 or the human chorionic myelogenous leukemia cell line K562) or HLA-transfected insect cells, as described in Britten et al, Journal of Immunological Methods, (2002) 259:95; Latouche et al, Nature Biotechnology, (2000) 18:405; and Guelly et al, Eur. J. Immunol. (2002) 32:182, each of which is expressly incorporated by reference in their entirety. While such artificial antigen presenting cells appear to function relatively well in ELISpot assays for detection of T cell activity, such artificial antigen presenting cells may express undesired proteins on their surface (including other MHC molecules) and/or may incorrectly fold or denature the MHC molecules (such as observed with the expression of HLA heavy chains in insect cells). In addition, the use of these prior known antigen presenting cells as a vaccine would require thorough characterization of such cells. Therefore, the use of liposomes as artificial antigen presenting cells which may be utilized as vaccine candidates overcomes the disadvantages and defects of the prior art.

The recombinant, soluble MHC-peptide complex(es) are mixed with lipids to form liposomes. Liposomes have been studied for many years because of their structure and their potential use as drug delivery vehicles. Methods of forming liposomes are well known to those of ordinary skill in the art, and any of the standard liposome formation methods (such as that disclosed in Prakken et al, Nature Medicine (2000) 6:1406, which is expressly incorporated herein in its entirety by reference) may be utilized in the formation of the artificial antigen presenting cells of the present invention. Lipids that may be utilized in the methods of the present invention include phosphatidylcholine, dioleoyl phosphatidylcholine, phosphatidylethanolamine, phosphatidylinositol, phosphatidic acid, cholesterol, 1,2-dioleoyl-sn-glycero-3-[N-(5-amino-1-carboxypentyl)imidodiacetic acid)succinyl] (DOGS-NTA), and combinations thereof. When a histidine tail is utilized as the tag attached to the recombinant soluble MHC molecule, a nickel-chelating lipid may be utilized, such as DOGS-NTA(nickel salt).

The lipid molecules form a bilayered membrane, and the recombinant soluble MHC-peptide complex is anchored to such membrane by the tag attached thereto. Celia et al (PNAS (1999) 96:5634), the Specification of which is hereby expressly incorporated herein by reference, demonstrates that a nickel-chelating lipid allows capture and proper orientation of histidine-tagged MHC molecules on the surface of liposomes or lipid monolayers, such that T cell receptor binding can be observed.

At least one additional signal molecule may also be mixed with the lipids and the recombinant, soluble MHC-peptide complexes for incorporation in the liposome. Such signal molecules act to manipulate the intensity and quality of the T cell response by encouraging interactions with other specific cells and thereby directing certain immune responses. For example, if the antigenic peptide of interest is a peptide that distinguishes an infected cell from an uninfected cell, additional coreceptors such as CD54 (ICAM-1), CD80 (B7.1), CD86 (B7.2), CD58 (LFA-3) and/or CD28 receptor may be incorporated in the aAPC which activate other components of the immune response, providing a heightened state of reaction to the antigenic peptide. The coreceptors may modulate the immune response down another path by activating different T Helper cells, such as T_{H1} or T_{H2}, or by activating different subclasses of antibody, such as IgA, IgD, IgE, IgG or IgM. In addition, another MHC molecule may be incorporated therein to act as an allogeneic adjuvant and heighten the immune response. Alternatively, if the antigenic peptide of interest is actually a self peptide to which an autoimmune response has been observed or a peptide responsible for a rejection response in transplantation, additional coreceptors may be incorporated in the aAPC which down regulate the immune response or activate a different response pathway. Examples of such molecules include CTL4A and Fas ligand.

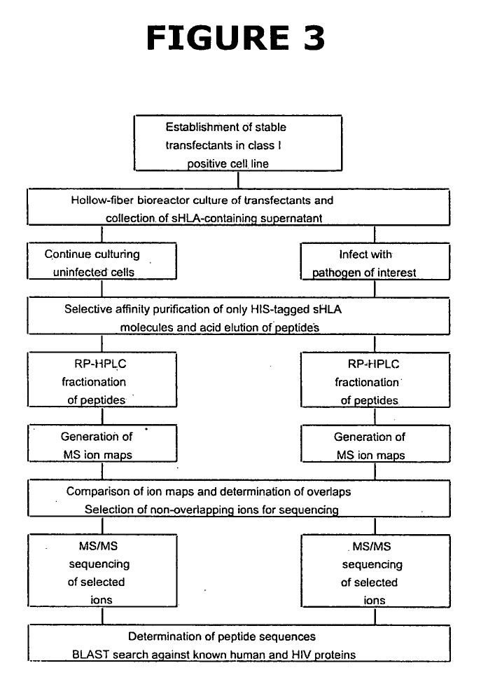

The recombinant, soluble MHC-peptide complex incorporated in the artificial antigen presenting cell includes a desired peptide of interest bound to the antigen binding groove of the recombinant soluble MHC molecule. The desired peptide of interest may be identified by the method of epitope discovery described in U.S. Ser. No. 09/974,366, filed Oct. 10, 2001, entitled “COMPARATIVE LIGAND MAPPING FROM MHC POSITIVE CELLS”, the Specification of which is hereby expressly incorporated herein by reference in its entirety. The method disclosed and claimed in U.S. Ser. No. 09/974,366 identifies and isolates at least one peptide that distinguishes an infected or tumor cell from an uninfected or nontumor cell. Such method is outlined in FIG. 3 and further utilizes the method of producing recombinant, soluble MHC molecules described herein and utilized in the method of the present invention. Briefly, a suitable host cell containing a construct encoding the recombinant, soluble MHC molecule is infected with at least one of a microorganism, a gene from a microorganism, or a tumor gene, and the secreted recombinant, soluble MHC molecules are purified and their peptide cargo isolated and compared to the peptide cargo isolated from an uninfected host cell also containing the construct encoding the recombinant, soluble MHC molecule. In addition, such method described in U.S. Ser. No. 09/974,366 would allow for isolation of the recombinant, soluble MHC molecule-peptide complex of the present invention.

Alternatively, the peptide may have been identified by other methods of epitope discovery and testing for immunogenicity (including the method of epitope testing described in provisional application U.S. Ser. No. 60/274,605, filed Mar. 9, 2001, entitled “EPITOPE TESTING USING SOLUBLE HLA”, the Specification of which is hereby expressly incorporated in its entirety by reference. When a peptide has been identified by other methods and it is desired to have such peptide complexed with the recombinant soluble MHC molecules produced by the method described herein, a host cell defective in peptide processing may be utilized. Such host cell will not produce endogenous peptides for loading into MHC molecules and display on the cell surface. The desired peptide may then be produced synthetically and pulsed into the host cell containing the construct encoding the recombinant, soluble MHC molecule so that the desired peptide can be loaded into the antigen binding groove of the recombinant soluble MHC molecule, thereby forming the recombinant soluble MHC-peptide complex for incorporation into the liposome. Optionally, a vector encoding the desired peptide may be introduced into the suitable host cell containing the construct encoding the recombinant, soluble MHC molecule so that the host cell expresses both the recombinant, soluble MHC molecule and the peptide, and the peptide is naturally loaded into the antigen binding groove of the recombinant soluble MHC molecule, thereby forming the recombinant soluble MHC-peptide complex for incorporation into the liposome.

One of the primary advantages of the present invention is the production of the recombinant soluble MHC molecules in such a manner that they are folded naturally and are trafficked through the cell in such a way that they are identical in functional properties to a native MHC molecule. Another primary advantage of the methods of the present invention is the isolation of MHC-peptide complexes containing peptides that are produced using the native host cell's machinery and that are loaded in MHC using the native host cell's machinery. This ensures that the recombinant soluble MHC-peptide complexes of the present invention will be recognized by the immune system. As the complexes described herein mimic antigen presenting cells while being free of any deleterious molecules that may have undesired effects, the complexes of the present invention are ideal vaccine candidates.

The identification of peptides that distinguish an infected or tumor cell from an uninfected or nontumor cell and incorporation of such peptide into the recombinant soluble MHC-peptide complex that is further incorporated into a liposome to form an artificial antigen presenting cell provides an ideal candidate for vaccination against infection by such pathogen or prevention of tumor formation. In addition, following infection or tumor formation, the above described artificial antigen presenting cell may further have at least one costimulatory molecule incorporated therein to heighten the immune response and target such infected or tumorigenic cells for destruction.

The importance of the utilization of natural peptide processing and loading in the methods of the present invention are clearly evident when the desired peptide complexed with the MHC is derived from an endogenous protein that is upregulated or trafficked differently upon infection or transformation of a cell. Such peptides would not be identified by the prior art methods of epitope discovery.

While the present invention has been described in detail with reference to the use of a liposome, other spherical molecules that comprise a bilayer and mimic the structure of a cell without containing the deleterious molecules found on the surface of a cell may be utilized in accordance with the present invention. For example, the present invention further envisions the use of molecules such as spheres formed from latex, polystyrene, or plastic beads. Although these molecules might not cap the way a lipid bilayer would, they could be coated with sufficient sHLA to make capping irrelevant

Thus, in accordance with the present invention, there has been provided a methodology for presentation of antigenic peptides utilizing artificial antigen presenting cells having recombinant, soluble MHC molecules incorporated therein, such methodology including methods for producing and manipulating Class I and Class II MHC molecules from gDNA that fully satisfies the objectives and advantages set forth herein above. Although the invention has been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the invention.

1. A complex, comprising:
a liposome;
at least one recombinant soluble MHC-peptide complex, comprising:
a recombinant soluble MHC heavy chain molecule containing a tag for anchoring the recombinant soluble MHC-peptide complex to the liposome;
a beta-2-microglobulin molecule native to and endogenously expressed in a host cell having a construct encoding the recombinant soluble MHC heavy chain molecule, wherein the beta-2-microglobulin is associated with the recombinant soluble MHC heavy chain molecule in the host cell; and
an endogenously produced peptide bound to an antigen binding groove of the recombinant soluble MHC heavy chain molecule, wherein the endogenously produced peptide is loaded into the antigen binding groove of the recombinant soluble MHC heavy chain molecule in the host cell; and
wherein the at least one recombinant soluble MHC-peptide complex is incorporated into the liposome such that the at least one recombinant soluble MHC-peptide complex is available to bind a T cell receptor on a T cell, thereby activating or suppressing the T cell.

2. The complex of claim 1 wherein the recombinant soluble MHC molecule is a Class I MHC molecule or a Class II MHC molecule.

3. The complex of claim 1 further comprising at least one additional signal molecule incorporated in the liposome for manipulating intensity and quality of the T cell response.

4. The complex of claim 1, wherein the at least one recombinant soluble MHC-peptide complex is produced by a method comprising the steps of:
obtaining gDNA encoding a desired MHC heavy chain molecule;
creating a PCR product encoding a soluble form of the desired MHC heavy chain molecule by PCR amplification of the gDNA, wherein the PCR amplification utilizes at least one locus-specific primer, wherein the PCR product does not encode the cytoplasmic and transmembrane domains of the desired MHC heavy chain molecule, thereby producing a PCR product that encodes soluble MHC heavy chain molecule;
inserting the PCR product into a mammalian expression vector to form a construct that encodes the soluble MHC heavy chain molecule;
introducing the construct into at least one suitable host cell;
culturing the at least one suitable host cell under conditions that allow for expression of the soluble MHC heavy chain molecule from the construct, thereby producing soluble MHC complexes having the desired MHC heavy chain molecule associated with native beta-2-microglobulin and loaded with endogenously produced peptides, and wherein the recombinant soluble MHC heavy chain molecules are folded naturally and are trafficked through the cell in such a way that they are identical in functional properties to a native MHC heavy chain molecule expressed from the MHC allele and thereby associate with native beta-2-microglobulin and bind peptide ligands in an identical manner as full-length, cell-surface-expressed MHC heavy chain molecules; and
isolating the recombinant soluble MHC-peptide complexes.

5. The complex of claim 4 wherein, in the step of obtaining gDNA which encodes a desired MHC heavy chain molecule, the gDNA is obtained from blood, saliva, hair, semen, or sweat.

6. The complex of claim 4 wherein, in the step of creating a PCR product, the at least one locus-specific primer is a 3′ primer having a stop codon incorporated therein.

7. The complex of claim 4 wherein, in the step of creating a PCR product, the locus-specific primer includes a sequence encoding the tag such that the soluble MHC molecule encoded by the PCR product contains the tag attached thereto that also facilitates in purification of the soluble MHC molecules produced therefrom as well as anchoring the recombinant soluble MHC molecule to the liposome.

8. The complex of claim 7 wherein the tag is a histidine tail.

9. The complex of claim 8 wherein nickel is disposed in the liposome such that the interaction between the nickel and the histidine tail maintains the recombinant soluble MHC molecule in an anchored position on the liposome.

10. The complex of claim 7 wherein the tag is a biotinylation signal peptide.

11. The complex of claim 10 wherein the recombinant soluble MHC molecule containing the biotinylation signal peptide is biotinylated, and streptavidin is disposed in the liposome such that the interaction between biotin and the streptavidin maintains the recombinant soluble MHC molecule in an anchored position on the liposome.

12. The complex of claim 4 wherein, in the step of introducing the construct into at least one suitable host cell, the suitable host cell lacks expression of Class I MHC molecules.

13. The complex of claim 4 wherein, in the step of introducing the construct into at least one suitable host cell, the construct is electroporated into the at least one suitable host cell.

14. The complex of claim 4 wherein, in the step of introducing the construct into at least one suitable host cell, the construct is transfected into the at least one suitable host cell.

15. The complex of claim 4 wherein, in the step of introducing the construct into at least one suitable host cell, the suitable host cell is defective in peptide processing such that peptides are not formed for loading into MHC molecules.

16. The complex of claim 15 wherein the method of producing the at least one recombinant soluble MHC-peptide complex further comprises the step of introducing a construct encoding a desired peptide into the at least one suitable host cell such that the desired peptide expressed by the construct binds to the antigen binding groove of the recombinant soluble MHC molecule, thereby forming the recombinant soluble MHC-peptide complex.

17. An artificial antigen presenting cell, comprising:
a spherical molecule having a bilayer;
at least one recombinant soluble MHC-peptide complex, comprising:
a recombinant soluble MHC heavy chain molecule containing a tag for anchoring the recombinant soluble MHC-peptide complex to the spherical molecule;
a beta-2-microglobulin molecule native to and endogenously expressed in a host cell having a construct encoding the recombinant soluble MHC heavy chain molecule, wherein the beta-2-microglobulin is associated with the recombinant soluble MHC heavy chain molecule in the host cell; and
an endogenously produced peptide bound to an antigen binding groove of the recombinant soluble MHC heavy chain molecule, wherein the endogenously produced peptide is loaded into the antigen binding groove of the recombinant soluble MHC heavy chain molecule in the host cell; and
wherein the at least one recombinant soluble MHC-peptide complex is attached to the spherical molecule via interactions between the tag and the bilayer such that the at least one recombinant soluble MHC-peptide complex is available to bind a T cell receptor on a T cell, thereby activating or suppressing the T cell.

18. complex, comprising:
a liposome;
at least one recombinant soluble MHC-peptide complex, comprising:
a recombinant soluble MHC heavy chain molecule containing a tag for anchoring the recombinant soluble MHC-peptide complex to the liposome, the recombinant soluble MHC heavy chain molecule produced in a host cell having a construct encoding the recombinant soluble MHC heavy chain molecule therein;
a beta-2-microglobulin molecule native to and endogenously expressed in the host cell, wherein the beta-2-microglobulin is associated with the recombinant soluble MHC heavy chain molecule in the host cell; and
a peptide bound to an antigen binding groove of the recombinant soluble MHC heavy chain molecule, wherein the endogenously produced peptide is loaded into the antigen binding groove of the recombinant soluble MHC heavy chain molecule in the host cell, wherein the host cell is defective in peptide processing such that peptides are not formed for loading into MHC molecules, and the peptide is pulsed into the host cell; and
wherein the at least one recombinant soluble MHC-peptide complex is incorporated into the liposome such that the at least one recombinant soluble MHC-peptide complex is available to bind a T cell receptor on a T cell, thereby activating or suppressing the T cell.

19. The complex of claim 18 wherein the recombinant soluble MHC molecule is a Class I MHC molecule or a Class II MHC molecule.

20. The complex of claim 18 further comprising at least one additional signal molecule incorporated in the liposome for manipulating intensity and quality of the T cell response.

21. The complex of claim 18 wherein the tag is a histidine tail.

22. The complex of claim 18 wherein nickel is disposed in the liposome such that the interaction between the nickel and the histidine tail maintains the recombinant soluble MHC molecule in an anchored position on the liposome.

23. The complex of claim 18 wherein the tag is a biotinylation signal peptide.

24. The complex of claim 23 wherein the recombinant soluble MHC molecule containing the biotinylation signal peptide is biotinylated, and streptavidin is disposed in the liposome such that the interaction between biotin and the streptavidin maintains the recombinant soluble MHC molecule in an anchored position on the liposome.

25. The complex of claim 18 wherein, in the step of introducing the construct into at least one suitable host cell, the suitable host cell lacks expression of Class I MHC molecules.

26. The complex of claim 18 wherein, in the step of introducing the construct into at least one suitable host cell, the construct is electroporated into the at least one suitable host cell.

27. The complex of claim 18 wherein, in the step of introducing the construct into at least one suitable host cell, the construct is transfected into the at least one suitable host cell.

28. An artificial antigen presenting cell, comprising:
a spherical molecule having a bilayer;
at least one recombinant soluble MHC-peptide complex, comprising:
a recombinant soluble MHC heavy chain molecule containing a tag for anchoring the recombinant soluble MHC-peptide complex to the liposome, the recombinant soluble MHC heavy chain molecule produced in a host cell having a construct encoding the recombinant soluble MHC heavy chain molecule therein;
a beta-2-microglobulin molecule native to and endogenously expressed in the host cell, wherein the beta-2-microglobulin is associated with the recombinant soluble MHC heavy chain molecule in the host cell; and
a peptide bound to an antigen binding groove of the recombinant soluble MHC heavy chain molecule, wherein the endogenously produced peptide is loaded into the antigen binding groove of the recombinant soluble MHC heavy chain molecule in the host cell, wherein the host cell is defective in peptide processing such that peptides are not formed for loading into MHC molecules, and the peptide is pulsed into the host cell; and
wherein the at least one recombinant soluble MHC-peptide complex is attached to the spherical molecule via interactions between the tag and the bilayer such that the at least one recombinant soluble MHC-peptide complex is available to bind a T cell receptor on a T cell, thereby activating or suppressing the T cell.
